(19) United States
(12) Patent Application Publication
(10) Pub. No.: US 2004/0175329 A1
(43) Pub. Date: Sep. 9, 2004

(54) Method for continuous visualization of a body lumen

(76) Inventor: John Steele Fisher, Belleair, FL (US)

Correspondence Address: Paul D. Greeley, Esq., Ohlandt, Greeley, Ruggiero & Perle, L.L.P., One Landmark Square, 10th Floor, Stamford, CT, 06901-2682, US

(21) Appl. No.: 10/383,796

(22) Filed: Mar. 7, 2003

(51) Int. Cl. A61K 51/00; A61K 49/00

(52) U.S. Cl. 424/9.36

(57) ABSTRACT

There is disclosed a method for visualizing a body lumen. The method of the invention uses contrast material that preferably adheres to the interior wall of a target body lumen for a period of time sufficient to permit real-time visualization of the lumen and to enable the performance of image-guided diagnostic and therapeutic procedures. Lumens from the cardio-vascular, pulmonary, digestive, reproductive, excretory and central nervous systems may conveniently be visualized. Radiography, nuclear medicine, ultrasound and MRI visualizing systems may be used. Contrast materials for use in the invention are non-toxic, especially non-toxic to the kidney, do not produce allergic reactions, do not stimulate atherogenesis, and preferably comprise iodine or a metal.

### BACKGROUND OF THE INVENTION

1. Field of the Invention

The present invention relates to diagnostic imaging contrast compositions and method of using such compositions. More particularly, the present invention relates to imaging compositions that adhere for a period of time to body lumen walls, especially those of the circulatory system, and provide extended visualization for invasive medical diagnostic and therapeutic procedures.

2. Description of the Prior Art

Contrast agents have long been used in a variety of medical imaging procedures to enhance the contrast of patient images. Contrast agents or media containing contrast agents may, for example, be employed with x-ray, magnetic resonance and ultrasound imaging. Such imaging procedures involve visualization of lumens, such as blood vessels in cardiac angiography, either by x-ray imaging or by magnetic resonance imaging (MRI), intravenous urography or kidney imaging, computerized tomography, neurological visualization of the central nervous system (i.e., the spinal cord, brain etc.), the digestive tract, lymphatics, bronchi, biliary ducts and the like. Imaging procedures are widely used in the practice of contemporary medicine. There are more than 10 million x-ray radiological examinations involving the use of contrast media performed each year in the United States and the number is growing. It is estimated that approximately 5-10% of these procedures are accompanied by clinical side effects with life threatening complications occurring in a portion of such procedures. The use of any particular contrast medium is related to its diagnostic efficacy, its toxicity, its ease of storage and administration, and by consideration of adverse effects it may have on the patient to which it is administered. It is desirable to have contrast media that are effective and have as few as possible deleterious physiological effects on body cells or organs.

In medical procedures involving the use of contrast media, there are several undesirable side effects including hyperosmotic damage, iodine-specific toxicity, kidney damage, and radiation damage. As an example, it is typical to inject 100-200 mL of contrast medium into a total plasma volume of 5 liters within a period of several minutes. Cells, such as endothelial cells, red blood cells, white blood cells, kidney cell etc., are exposed to a hyperosmotic solution in comparison to the osmolarity of the blood. This gives rise to hyperosmotic shock which may produce damage. This hypertonicity causes osmotic effects, such as the draining of water from red blood cells, endothelia cells, and heart and blood vessel muscle cells. Hypertonicity, chemotoxicity, and non-optimal ionic composition either individually or collectively reduce the contractile force of the muscle cells and cause dilation of small blood vessels and result in a decrease in blood pressure.

Iodine is commonly used in contrast media. For example, in an x-ray visualization procedure typically 30-40 grams of iodine/contrast medium are injected into the blood within a period of about 2-10 minutes. Visualization of a target requires a minumum accumulation of 15-20 mg of iodine/ml in the target tissue. For this reason, the initial iodine concentration of the contrast medium is relatively high (i.e., in the range of about 300 to 420 mg iodine/ml of medium). Iodinated aromatic compounds may be used as x-ray contrast agents. U.S. Pat. No. 6,406,680 is directed to iodinated alkenes for use as x-ray contrast agents reported as equivalent to iodinated aromatic contrast agents. Compounds that can release iodide in reactions with nucleophiles or electrophiles may cause toxic biological effects and preferably should not be used as contrast agents.

As iodine is a common contrast agent, the iodine load to which the kidney is exposed and needs to excrete is a potential cause for renal damage. In general, it is believed that about 12% of all patients that are injected with an x-ray contrast medium encounter renal complications. In cardiac catheterization, for example, from 9-16% of patients develop renal failure depending upon whether they are high or low risk patients. It is well known that exposure of cells to x-ray contrast medium causes cell damage. In addition, with commercial x-ray contrast media having high concentrations of iodine of about 300 mg iodine/mL, these media have a relatively high viscosity at ambient temperature. Such high viscosity is troublesome to the provider of the contrast medium and requires relatively large bore needles or high-applied pressure. This is particularly significant in pediatric radiography and in radiographic techniques, such as angiography, which require rapid bolus administration. Although the toxicity of iodine as a contrast agent is notable, toxicity and adverse biological effects of a contrast medium are attributed to the components of a the medium, such as the solvent or carrier, as well as the contrast agent itself and its components (i.e., ions if ionic) and various metabolites.

Coronary angiography is an important procedure in the diagnosis of medical problems associated with the coronary arteries that supply blood to the heart. During this procedure, the coronary arteries are imaged so as to enable the medical practitioner to observe any blood circulation problems that may affect the heart. A radio-opaque contrast substance (i.e., contrast medium with iodine as the active contrast agent) injected into the coronary arteries during the angiography procedure causes the arteries to appear as bright lines contrasted against a relatively darker background. Where a stenosis or restriction is present in a coronary artery, the artery will appear to be pinched and will have a smaller cross-sectional thickness at the location of the restriction. It is typically necessary to produce at least five angiography sequences at different projection angles relative to the heart to obtain visual images of all portions of the coronary arterial system for accurate medical diagnosis.

During angiography, a radio-opaque contrast substance is injected into one of the coronary arteries and consecutive frames (i.e., from about 150 to 250) are recorded on film with a cine camera, video camera, and/or recorded in digital format. Multiple injections or sequences are usually involved with an angiography procedure. Each sequence records from 5 to 15 heartbeats or cardiac cycles. During each beat of the heart, ventricles fill with blood during diastole and reach their maximum volume at the end of diastole. The heart muscle then contracts during the systole phase, and the ventricles reach their minimum volume of blood at the end of systole. The filling of the coronary arteries with blood takes place primarily during diastole as the coronary arteries pass through the heart muscle and the pressure exerted by the contracting muscle during the systole phase tends to impede blood flow through the arteries. During the imaging procedure, the injected radio-opaque contrast substance can be seen to fill the coronary artery and then to gradually clear from the artery as fresh blood devoid of the contrast substance enters the artery.

Contrast media used in coronary angiography are injected into the circulatory system and have been associated with several serious adverse effects on cardiac function. In this procedure following injection of the contrast medium, a bolus of the contrast medium rather than blood flows through the circulatory system. Differences in the chemical and physical nature of the contrast medium and the blood that it displaces temporarily may produce undesirable side effects, such as arrhythmias, reduction in cardiac contractile force, ventricular fibrillation and the like. Accordingly, it is a desirable objective to reduce such negative effects on cardiac function from the infusion of contrast media into the circulatory system during angiography and other similar procedures. In MRI methodology for visualization of blood vessels, a paramagnetic substance dissolved in a hyperosmotic contrast medium is injected. Factors contributing to contrast media toxicity are chemotoxicity of the contrast agent, osmolalilty of the contrast medium, and the ionic/non-ionic composition of the contrast medium.

Coronary artery disease is currently the leading cause of death in the Western Hemisphere. Accordingly, visualization of the coronary arteries is a critical step in the diagnosis, treatment and prevention of death and disability from this disease. While angiography, the mapping of blood vessels, is performed with a number of techniques, the most commonly employed procedures involve invasive techniques (i.e., x-ray angiography, nuclear medicine, or surgery). Angiography commonly involves the injection for contrast of an x-ray opaque dye into the patient, allowing a period of time to pass in order to permit the dye to become circulated within the blood stream, and thereafter exposing the patient to ionizing radiation (e.g., x-rays) in order to image the patient's blood vessels. In x-ray radiography, a catheter for injection of contrast material is inserted into the artery through the groin area of a patient. Passive non-invasive techniques such as magnetic resonance imaging may also be employed. U.S. Pat. No. 6,265,875 is directed to a method of MRI tissue differentiation.

The various visualization techniques each have their disadvantages. For example, x-ray techniques expose both the patient and provider to dangerous ionizing radiation in order to image blood vessels of the patient. While in general exposure to x-rays (i.e., ionizing radiation) is preferably avoided, it is particularly undesirable in certain circumstances (i.e., pregnancy). Angiography generally requires high contrast between tissue and blood vessels in a patient to visualize blood vessels. In MRI systems, the patient must remain still for an extended period of time and expensive equipment is required. There is need for a methodology which allows angiography and other lumen visualizations without some of the disadvantages of conventional systems.

Other adverse side effects are associated with the use of contrast media. For example, patients often experience discomfort. Such discomfort is very commonly in the form of a burning sensation, experienced when the contrast medium is injected and subsequent to the injection. The severity and duration of such discomfort increases as the amount of contrast medium injected is increased. Also, contrast media may adversely affect a patient's kidneys. The extent of the effect of the contrast media on the patient's kidneys will depend on the patient's renal health and the amount and type of contrast media used. Contrast media generally fall into two general categories: (i) ionic contrast media; and (ii) non-ionic contrast media. In these groups, the contrast agent in a carrier fluid is either in ionic form or in molecular or particulate form. In general, it is advisable to minimize the amount of contrast media employed. The amount of contrast medium used should be the smallest or minimal amount needed to provide diagnostically useful images of targets.

U.S. Pat. No. 5,394,874 is directed to angiography using ultrasound. Pulse echo ultrasonic imaging technology is used for examining the internal structure and functioning of living organisms, including blood flow. In medical diagnoses of various conditions, it is useful to examine soft tissues and/or blood flow to show structural details of body organs and vessels in the organs. In the examination of internal body structures, ultrasonic images are formed by producing very short pulses of ultrasound using a transducer, sending the pulses through the body, and thereafter measuring the properties of the echoes (e.g., amplitude and phase) from targets at various depths within the body. Typically, the ultrasound beam is focused at various depths within the body in order to improve resolution or image quality. A transducer receives the echoes, typically the same transducer used for transmission, and processed to generate an image of the target. Measuring and imaging blood flow, or other fluid flow, in the human body is typically done using the well-established Doppler principle, where a transmitted burst of ultrasound at a specific frequency is reflected from moving blood cells, thereby changing the frequency of the reflected ultrasound in accordance with the velocity and direction of the flow.

Regardless of the radiological imaging system (i.e., magnetic resonance imaging, computed tomography, or conventional radiograpahy using x-ray) or the part of the body being imaged, contrast-enhancing compositions are quite useful and widely employed by medical professionals. The use of contrast agents as adjuncts in radiological imaging makes it possible to determine the location, size, and conformation of organs or other structures of the body relative to surrounding tissues or structures. The various imaging systems, including radiological and sound systems, operate on distinct physical principles, and each may be used to differentiate between normal tissue, tumors, lesions, blockages and the like, but all may employ contrast agents. For example, in the diagnosis of disorders of the gastrointestinal (GI) tract, it is difficult to identify blockages or abnormalities in the conformation of the intestine unless the particular section of the GI tract under investigation is filled with a contrast agent which facilitates definition of volumes and delineation of boundaries.

In conventional radiography, a beam of x-rays passes through a target and exposes an underlying photographic film thereby providing a visual image. The developed film gives an image of the radiodensity pattern of the target object. Less radio-dense areas show a blackening of the film and more radio-dense area (i.e., bone) produce a lighter image. Contrast agents for use with x-ray radiography may be either less or more radiodense than body tissues. Examples of less radio-dense contrast agents include air or other gases (i.e., carbon dioxide for use in the GI tract). Examples of more radio-dense contrast material included iodine compositions, barium sulfate suspensions, clay-based compositions, and the like. U.S. Pat. No. 3,975,512 is directed to the use of fluorocarbons as contrast enhancement media in radiological imaging. Depending on the imaging requirement, contrast agents are introduced into the body in various ways (i.e., orally with the GI tract; injection with coronary angiography). Regardless of the imaging system, a suitable contrast agent must be biocompatible. Contrast agents should be non-toxic, chemically stable, should not be absorbed by the body or reactive within tissue, and should be safely eliminated from the body within a short period of time.

With reference to magnetic resonance imaging (MRI), a different physical principle is employed. MRI takes advantage of the fact that some atomic nuclei (e.g., hydrogen nuclei) have both nuclear spin and nuclear magnetic moment can therefore be manipulated by applied magnetic field. In conventional MRI systems, a magnetic field is established across a body to align the spin axes of the nuclei of a particular chemical element, usually hydrogen, with the direction of the magnetic field. The aligned spinning nuclei execute motions around the aligning direction of the magnetic field. The frequency at which the aligned spinning nuclei process around the direction of the magnetic field is a function of the particular nucleus which is involved and the strength of the magnetic field. In commercial MRI systems following alignment or polarization of the selected nuclei, a burst of radio frequency energy at the resonant frequency is radiated at the target object to produce a coherent deflection of the spin alignment of the selected nuclei. When the deflecting radio energy is terminated, the deflected or disturbed spin axes are reoriented or realigned and radiate a characteristic radio frequency signal which can be detected and analyzed. The MRI system can establish image contrast between different types of tissues in the body. A wide variety of different excitation and discrimination modes are known in the art. Accordingly, contrast agents for MRI must possess a substantially different concentration of the nuclei used as a basis for scanning. In a hydrogen scanning system, an imaging agent substantially lacking hydrogen can be used. In a MRI system scanning for a physiologically minor nucleus, e.g., fluorine nuclei, an imaging substance with a high concentration of hydrogen would provide appropriate contrast.

While MRI utilizes radio frequency pulses and magnetic field gradients applied to a patient in a strong field to produce visual images, contrast agents are used to improve magnetic resonance images. Such contrast agents include magnetizable substances having metals or metallic compounds. Such contrast agents may be paramagnetic, ferromagnetic, or supermagnetic and act through dipole interactions with tissue protons. Most magnetic resonance imaging contrast agents have similar mechanisms of action. Most are based on gadolinium chelates and are paramagnetic agents that develop a magnetic moment when placed in a magnetic. Magnetic resonance contrast agents are increasingly being used for magnetic resonance angiography. Both arterial and venous signals become equally enhanced. With current contrast agents, there is difficulty eliminating either arterial or venous signals for flow discrimination. Automatic bolus detection addresses this issue when the blood flow is in the arterial phase. However, with the contrast agent in place, subsequent data must contend with the increased venous signal intensity as the contrast agent continues to distribute in the system. In addition, it is expected that that the use of intravascular contrast agents with much longer persistence will require more novel techniques for arterial-venous discrimination. U.S. Pat. No. 6,192,264 is directed to a method for MRI venography including arterial and venous discrimination. Phase contrast magnetic resonance angiography is used for imaging blood flow.

Following heart disease and cancer, the most common cause of death in the United States is cerebrovascular disease. The most common cerebrovascular pathologies are: (i) stenoses or narrowing due to vessel degeneration; (ii) aneurysms or bulges; and (iii) arteriovenous malformations which act as short circuits. Hemorrhage and other incidents attributable to these pathologies or acute thrombogenesis leading to vessel constriction or blockage can led to stroke resulting in death or devastating disabilities. Diagnostic imaging as well as therapeutic image-guided procedures are used in the treatment of cerebrovascular diseases.

In the past, the treatment of choice for vascular disease was invasive surgery that inherently carries substantial risks. More recently, image-guided minimally invasive endovascular treatments are becoming increasingly preferred for medical treatment. Such endovascular treatments are primarily radiographically related procedures. As new procedures are developed involving smaller and smaller catheters and devices, great importance is placed on image quality. There is a growing requirement for high spatial resolution during endovascular interventions or treatment. For example, balloon expansion of a stent or attempts to mold the stent within the treated vessel depend upon images with adequate detail. Visualizing the spatial relationship between overlapping stents, where required, is difficult. Also, detecting the movement of stents during the placement process is challenging. With newer stents having smaller gauge wire and more complex design, it is becoming very difficult to see even the gross shape of the stent, let alone to determine the status of the individual segment or wires of such devices. As endovascular devices progress toward treatment of smaller vessels (i.e., within or beyond the Circle of Willis) there will be the additional concern about disturbing or blocking the origin or perforators. These perforators are micro in size and are often extremely important vessels for specific, key neurological functions, which if blocked can produce devastating effects in the patient. Perforators seen during invasive microsurgery typically cannot be visualized easily, if at all, during conventional image-guided endovascular procedures. For aneurysm treatment with detachable coils, the thin strands of overlapping coils are typically blurred together into a dense mass with standard equipment. Visualization of the detailed shape of the aneurysm and the location and spacing of coil loops could determine the success or failure of the treatment.

### SUMMARY OF THE INVENTION

A method for visualizing, preferably continuously visualizing, a body lumen comprising: applying contrast material to the body lumen, wherein the contrast material adheres completely or in part to the wall of the body lumen; and visualizing the body lumen continuously over a period time by a visualizing system. The target body lumen is selected from the group consisting of: the cardio-vascular system, the pulmonary system, the digestive system, the central nervous system, the reproductive system, and the excretory system. Preferred body lumens are arteries, veins, capillaries and lymphatic vessels. The contrast material is preferably an ionic or non-ionic contrast material comprising at least one heavy atom (i.e., having an atomic weight of about 30 or greater). An embodiment of the invention comprises a contrast material having at least one metal. Contrast materials are selected from metals, paramagnetic materials, high atomic number non-metal materials, radioisotopes, gases or gas precursors, chromatophores, fluorophores, electrical impedence materials, and any combinations thereof. A particularly preferred contrast material comprises iodine. Continuous visualization preferably occurs over a period of time sufficient to permit the performance of image-guided medical procedures selected form the group consisting of: diagnostic procedures, therapeutic procedures and any combinations thereof. Preferred procedures are selected from the group consisting of: manipulation of wires, manipulation of catheters, and manipulation of stents.

The visualizing system of the invention may be a radiography system, a nuclear medicine system, an ultrasound system, a magnetic resonance system or any combinations thereof. The contrast material optionally comprises an endothelial binding substance. Contrast materials have the following characteristics: (i) adherence to the interior wall of a target body lumen for at a period of time sufficient to perform invasive image-guided procedures selected from the group consisting of: diagnostic procedures, therapeutic procedures, and any combinations thereof, wherein the target body lumen is selected from the group consisting of: the cardio-vascular system, the pulmonary system, the digestive system, the central nervous system, the reproductive system, and the excretory system; (ii) provides visibility of the body lumen by a visualizing system sufficient to perform said image-guided diagnostic procedures, wherein the visualizing system is selected from the group consisting of: radiography systems, nuclear medicine systems, ultrasound systems, magnetic resonance systems and any combinations thereof; and (iii) exits from the body without causing either kidney toxicity, allergic reaction, or stimulation of atherogenesis. The contrast material preferably biodegrades. The contrast material may further have endothelial binding capability. The contrast material adheres to the target lumen wall, and preferably, but not necessarily limited to, the interior lumen wall. Adherence of contrast material should last for a period of time from about 1 second to about 1 hour or more, preferably from about 10 seconds to 5 minutes, and more preferably from about 15 seconds to about 2 minutes.

The invention provides a method for selectively binding contrast material to a lumen wall comprising: administering an effective amount of a contrast material of the invention to a lumen, wherein the contrast material adheres for a period of time to a lumen wall; and visualizing the lumen by a visualizing system.

### DETAILED DESCRIPTION OF THE INVENTION

The present invention involves coating the interior wall of a lumen with contrast imaging material, which adheres preferably to the interior surface of the lumen wall for a period of time sufficient to permit visualization for performance invasive image-guided medical procedures, including diagnostic procedures, therapeutic procedures, and combined diagnostic and therapeutic procedures. Compositions for use in the present invention may be a contrast agent or a material containing a contrast agent in combination with another ingredient or several ingredients. Imaging may be effected by radiography, nuclear medicine, ultrasound, or magnetic resonance systems. Compositions of the invention are not toxic to the renal system or other system of the body, are not allergenic, and do not stimulate atherogenesis.

The present invention provides a method of imaging any of the mammalian or other animal lumens, including those of the circulatory system (i.e., arteries, veins, capillaries, and lymphatics), the pulmonary system (i.e., bronchi), the central nervous system (i.e., the spinal cord, brain, and nerves), the digestive system (small and large intestines, colon, liver, and bile ducts), the excretory system (kidneys, bladder and urological ducts), and the reproductive system (i.e., the uterus). Imaging may be done by any convenient system, including nuclear medicine imaging, preferably x-ray, magnetic resonance imaging (MRI). Cardiac angiography is a particularly preferred embodiment.

The term “contrast imaging agent” refers to any composition or material in any chemical form that is detectable in a diagnostic imaging procedure. Contrast agents may be organic or inorganic and are commonly metals or metal complexes or non-metals with high atomic weight (i.e., iodine). Iodinated contrast agents are a preferred embodiment. Contrast imaging material for use in the invention must efficaciously adhere, completely or in part, to the target lumen wall long enough to permit real time imaging sufficient to allow diagnosis of a medical condition and/or the performance an invasive medical procedure.

The active detection component of the contrast agents of the invention may be any material capable of detection either directly or indirectly in an in vivo diagnostic imaging procedure. Suitable materials are those which emit or may be caused to emit detectable radiation (i.e., by radioactive decay, fluorescence excitation, spin resonance excitation, etc.), materials which affect local electromagnetic fields (i.e., paramagnetic, superparamagnetic, ferrimagnetic or ferromagnetic species), materials which absorb or scatter radiation energy (i.e., chromophores; particles, including gas or liquid containing vesicles); heavy elements and compounds thereof, etc., and materials which generate a detectable substance (i.e., gas micro-bubble generators or the like).

A variety of materials detectable by diagnostic imaging may be employed. The preferred contrast agent should be selected according to the desired imaging procedure. For example, with ultrasound imaging an echogenic material, or a material capable of generating an echogenic material will normally be selected. With X-ray imaging, the contrast agent will generally be or contain a heavy atom, preferably having an atomic weight of about 30-38 or greater. With magnetic resonance imaging, the contrast agent will either be a non-zero nuclear spin isotope or a material having unpaired electron spins and hence having paramagnetic, superparamagnetic, ferrimagnetic or ferromagnetic properties. For light imaging, the contrast agent will be a light scatterer, either a colored or uncolored particle, a light absorber or a light emitter. With magnetometric imaging, the contrast agent will have detectable magnetic properties. With electrical impedance imaging, the contrast agent will affect electrical impedance. For scintigraphy, SPECT, PET or the like, the detected moiety will be a radionuclide.

Examples of contrast agent materials are, for example, magnetic iron oxide particles, gas-containing vesicles, and chelated paramagnetic metals (i.e., Gd, Dy, Mn, Fe etc.). See, for example, U.S. Pat. Nos. 4,647,447; 4,863,715; 4,770,183; 5,228,446; 5,387,080; 6,303,101; 6,404,680; 5,447,711; 6,420,436; 6,310,243; 6,448,442; 6,149,891, and 6,051,207. These patents are incorporated herein in their entirety.

Particularly preferred as reporting or contrast agents are: chelated para magnetic metal ions, such as Gd, Dy, Fe, and Mn, especially when chelated by macrocyclic chelant groups (e.g. tetraazacyclododecane chelants, such as DOTA, DO3A, HP-DO3A an d analogues thereof) or by linker chelant groups such as DTPA, DTPA-BMA, EDTA, DPDP, etc; metal radionuclides (i.e., such as those of Y, Tc, Sc, Ga, Cr, Sn, Cu, Tm, Ru, Re, Lu, Au, Pb and Ce); superparamagnetic iron oxide crystals; chromophores and fluorophores having absorption and/or emission maxima in the range between about 300 to 1400 nm, especially between about 600 nm to 1200 nm, most particularly between about 650 to 1000 nm; vesicles containing fluorinated gases (i.e., containing materials in the gas phase at 37° C. which are fluorine containing, chelated heavy metal cluster ions, e.g., W or Mo polyoxoanions or the sulphur or mixed oxygen/sulphur analogs); covalently bonded non-metal atoms which are either high atomic number (e.g., iodine) or are radioactive; iodinated compound containing vesicles; and the like.

In general, the active contrast, reporting, or detecting entity may be: (1) a chelatable metal or polyatomic metal-containing ion (i.e., TcO, etc), where the metal is a high atomic number metal (i.e., atomic number greater than about 30-37), a paramagentic species (i.e., a transition metal or lanthanide), or a radioactive isotope; (2) a covalently bound non-metal species which is an unpaired electron site (i.e., an oxygen or carbon in a persistent free radical), a high atomic number non-metal, or a radioisotope; (3) a polyatomic cluster or crystal containing high atomic number atoms, displaying cooperative magnetic behavior (e.g., superparamagnetism, ferrimagnetism or ferromagnetism) or containing radionuclides; (4) a gas or a gas precursor (i.e., a material or mixture of materials which is gaseous at 37° C.); (5) a chromophore (including fluorescent or phosphorescent material), e.g., an inorganic or organic structure, particularly a complexed metal ion or an organic group having an extensive delocalized electron system, or (6) a structure or group having electrical impedance varying characteristics.

Preferred contrast, reporting or detecting materials include chelated metal reporters including metal radionuclides, paramagnetic metal ions, fluorescent metal ions, heavy metal ions and cluster ions. Examples of referred metal radionuclides include ⁹⁰Y, ^{99M}Tc, 111In, ⁴⁷Sc, ⁶⁷Ga, ⁵¹Cr, ^{117m}Sn, ⁶⁷Cu, ¹⁶⁷Tm, ⁹⁷Ru, ¹⁸⁸Re, ¹⁷⁷Lu, ¹⁹⁹Au, ²⁰³Pb and ¹⁴¹Ce. Preferred paramagnetic metal ions include ions of transition and lanthanide metals (i.e., metals having atomic numbers of 6-9, 21-29, 42, 43, 44, or 57-71), in particular ions of Cr, V, Mn, Fe, Co, Ni, Cu, La, Ce, Pr, Nd, Pm, Sm, Eu, Gd, Tb, Dy, Ho, Er, Tm, Yb and Lu, more particularly of Mn, Cr, Fe, Gd and Dy, and especially Gd. Fluorescent metal ions for use in the invention include lanthanides, preferably La, Ce, Pr, Nd, Pm, Sm, Eu, Gd, Tb, Dy, Ho, Er, Tm, Yb, and Lu. Eu is especially preferred. Preferred heavy metal-containing materials may include atoms of Mo, Bi, Si, and W, and in particular may be polyatomic cluster ions (i.e., Bi compounds and W and Mo oxides).

The metal ions are desirably chelated by chelant groups in materials of the invention or on a particle (i.e., a vesicle or a porous or non-porous inorganic or organic solid), in particular linear, macrocyclic, terpyridine and N₂ S₂ chelants, such as DTPA, DTPA-BMA, EDTA, D03A and TaT. Additional examples are disclosed in U.S. Pat. Nos. 5,367,080 and 5,364,613, which are incorporated herein in their entirety. A chelator or chelating agent is a compound containing donor atoms that can combine by coordinate bonding with a metal atom to form a cyclic structure called a chelation complex or chelate. A chelant or chelating group may comprise the residue of one or more of a wide variety of cheating agents known in the art that can complex a metal ion or a polyatomic ion.

A suitable chelating agent can be selected from polyphosphates, such as sodium tripolyphosphate and hexametaphosphoric acid; aminocarboxylic acids, such as ethylenediaminetetraacetic acid, N-(2-hydroxy) ethylene-diaminetriacetic acid, nitrilotriacetic acid, N, N-di (2-hydroxyethyl) glycine, ethylene-bis (hydroxyphenylglycine) and diethylenetriamine pentacetic acid; 1,3-diketones, such as acetylacetone, trifluoroacetylacetone, and thenoyltrifluoroacetone; hydroxycarboxylic acids, such as tartaric acid, citric acid, gluconic acid, and 5-sulfosalicyclic acid; polyamines, such as ethylenediamine, diethylenetriamine, triethylenetetraamine, and triaminotriethylamine; aminoalcohols, such as triethanolamine and N-(2-hydroxyethyl)ethylenediamine; aromatic heterocyclic bases, such as 2,2′-diimidazole, picoline amine, dipicoline amine and 1,10-phenanthroline; phenols, such as salicylaldehyde, disulfopyrocatechol, and chromotropic acid; aminophenols, such as 8-hydroxyquinoline and oximesulfonic acid; oximes, such as dimethylglyoxime and salicylaldoxime; peptides containing proximal chelating functionality such as polycysteine, polyhistidine, polyaspartic acid, polyglutamic acid, or combinations of such amino acids; Schiff bases, such as disalicylaldehyde 1,2-propylenediimine; tetrapyrroles, such as tetraphenylporphin and phthalocyanine; sulfur compounds, such as toluenedithiol, meso-2,3-dimercaptosuccinic acid, dimercaptopropanol, thioglycolic acid, potassium ethyl xanthate, sodium diethyldithiocarbamate, dithizone, diethyl dithiophosphoric acid, and thiourea; synthetic macrocyclic compounds, such as dibenzo[18]crown-6, (CH₃)₆-[14]-4,11]-diene-N₄, and (2.2.2-cryptate); phosphonic acids, such as nitrilotrimethylene-phosphonic acid, ethylenediaminetetra(methylenephosphonic acid), and hydroxyethylidenediphosphonic acid, or combinations of two or more of the above agents. The residue of a suitable chelating agent preferably comprises a polycarboxylic acid group and preferred examples include: ethylenediamine-N,N,N′,N′-tetraacetic acid (EDTA); N,N,N′,N″,N″-diethylene-triaminepentaacetic acid (DTPA); 1,4,7,10-tetraazacyclododecane-N, N′,N″,N″′-tetraacetic acid (DOTA); 1,4,7,10-tetraazacyclododecane-N,N′,N″-triacetic acid (DO3A); 1-oxa-4,7,10-triazacyclododecane-N,N′,N″-triacetic acid (OTTA); and trans(1,2)-cyclohexanodiethylene-triamine-pentaacetic acid (CDTPA). Chelating agents may comprise proteins modified for the chelation of metals such as technetium and rhenium as described in U.S. Pat. No. 5,078,985, incorporated herein by reference.

Preferred chelating groups are 2-amiomethylpyridine, iminoacetic acid, iminodiacetic acid, ethylenediaminetetraacetic acid (EDTA), diethylenetriaminepentaacetic acid (DTPA), 1,4,7,10-tetraazacyclododecane-1,4,7,10-tetraacetic acid (DOTA), carbonyliminodiacetic acid, methyleneiminoacetic acid, methyleneiminodiacetic acid, ethylenethioethylene-iminoacetic acid, ethylenethioethyleneiminodiacetic acid, TMT, a terpyridinyl group, a chelating agent comprising a terpyridyl group and a carboxymethylamino group, or a salt of any of the foregoing acids. Especially preferred chelating groups are DTPA, DTPA-BMA, DPDP, TMT, DOTA and HPDO3A.

Methods for metallating chelating agents are within the level of skill in the art. Metals can be incorporated into a chelant moiety by direct incorporation, template synthesis and/or transmetallation. Direct incorporation is preferred. Metal ions can be easily complexed to chelating agent, for example, by merely exposing or mixing an aqueous solution of the chelating agent-containing moiety with a metal salt in an aqueous solution, preferably at a pH from about 4 to about 11. The salt can be any convenient salt, but preferably it is a water soluble salt of the metal, such as a halogen salt, and more preferably such salts are selected so as not to interfere with the binding of the metal ion with the chelating agent. The chelating agent-containing moiety is preferably in aqueous solution at a pH from between about 5 to about 9, more preferably a pH of between about 6 to about 8. The chelating agent-containing moiety can be mixed with buffer salts, such as citrate, acetate, phosphate and borate to produce the optimum pH. Preferably, the buffer salts are selected so as not to interfere with the subsequent binding of the metal ion to the chelating agent. In diagnostic imaging, containing a metal radionuclide, a ratio of metal radionuclide ion to chelating agent that is effective should be used. In preferred embodiments, the mole ratio of metal ion per chelating agent is between about 1:1,000 to about 1:1.

Contrast agents for use in the invention may be non-metal atomic materials or organic chromophoric or fluorophoric materials. Preferred non-metal atomic reporters include radioisotopes such as ¹²³I and ¹³¹I as well as non-zero nuclear spin atoms, such as ¹⁸F, and heavy atoms, such as I. The present invention preferably contemplates the use of radioisotopes of iodine. For example, if materials of the invention can be chemically substituted by iodine in a covalent bond forming reaction, such substituents can be labeled by methods well known in the art with a radioisotope of iodine. The iodine species can be used in diagnostic imaging applications. While, at the same time, a metal in a chelating agent can also be used in diagnostic imaging applications.

As with the metal chelants discussed earlier, contrast agents may be or carried in or on a vesicle or other particulate material.

Preferred organic chromophoric and fluorophoric reporters include groups having an extensive delocalized electron system, i.e., cyanines, merocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes and the like. Examples of chromophores, which may be used, include xylene cyanole, fluorescein, dansyl, NBD, indocyanine green, DODCI, DTDCI, DOTCI and DDTCI. Groups which have maximum absorption between about 600 and 1000 nm are preferred so as to avoid interference with hemoglobin absorption.

Additional examples of organic chromophoric or fluorophoric agents include, but are not limited to, cyanine dyes, chalcogenopyrylomethine dyes, pyrilium dyes, thiapyrilium dyes, squarylium dyes, croconium dyes, azulenium dyes, merocyanine dyes, indoaniline dyes including Cu and Ni complexes, indanthrene pigments, trisphenoquinone dyes, azo dyes, non-benzenoid aromatic dyes, tetrazine radical dyes, anthraquinone dyes, naphthoquinone dyes, metallated azo dyes including those containing Ni, Co, Fe and Mn, phthalocyanine dyes, naphthalocyanine dyes, metal phthalocyanines, metal naphthalocyanines, bis(dithiolene) metal complexes, bis(benzenedithiolate) metal complexes, bis(S,O-dithiolene) metal complexes, and tris(a-diimine) metal complexes. Representative examples are found in U.S. Pat. No. 6,051,207, which is incorporated herein in its entirety.

Examples of visible dyes include, but are not limited to, fluorescein derivatives, rhodamine derivatives, coumarins, azo dyes, metalizable dyes, anthraquinone dyes, benzodifuranone dyes, polycyclic aromatic carbonyl dyes, indigoid dyes, polymethine dyes, azacarbocyanine dyes, hemicyanine dyes, barbituates, diazahemicyanine dyes, stryrl dyes, diaryl carbonium dyes, triaryl carbonium dyes, phthalocyanine dyes, quinophthalone dyes, triphenodioxazine dyes, formazan dyes, phenothiazine dyes, such as methylene blue, azure A, azure B, and azure C, oxazine dyes, thiazine dyes, naphtholactam dyes, diazahemicyanine dyes, azopyridone dyes, azobenzene dyes, mordant dyes, acid dyes, basic dyes, metallized and premetallized dyes, xanthene dyes, direct dyes, leuco dyes which can be oxidized to produce dyes with hues bathochromically shifted from those of the precursor leuco dyes, and any other visible dyes known in the art.

Particulate visualization agents include those where the particle comprises a matrix or shell which carries or contains the agent and those where the particle matrix is itself the agent. Examples of the first category include vesicles (i.e., micelles, liposomes, micro-balloons and micro-bubbles) containing a liquid, gas or solid phase which contains the contrast effective reporter (i.e., an echogenic gas or a precursor therefor), a chelated paramagnetic metal or radionuclide, or a water-soluble iodinated X-ray contrast agent, porous particles loaded with the reporter, e.g., paramagnetic metal loaded molecular sieve particles; and solid particles (i.e., of an inert biotolerable polymer), onto which the agent is bound or coated (i.e., dye-loaded polymer particles).

Examples of the second category include, but are not limited to, light scattering organic or inorganic particles, magnetic particles (i.e., superparamagnetic, ferromagnetic or ferrimagnetic particles), and dye particles. Preferred particulate agents include superparamagnetic particles, echogenic vesicles, iodine-containing vesicles and dye-loaded polymer particles.

Non-peptidic endothelin receptors targeting vectors (such as, bosentan or BMS 182874) may be coupled directly or indirectly to a visualization agent, for example with covalently bound iodine radioisotopes, with metal chelates attached directly or via an organic linker group or coupled to a particulate agent (i.e., superparamagnetic crystals which are optionally coated), or a vesicle, (i.e., a gas containing or iodinated contrast agent containing micelle, liposome or micro-balloon). The contrast agents of the invention may be conveniently administered to patients for imaging in amounts determined by those skilled in the art that are sufficient to yield desired contrast with the chosen imaging system.

The visualization agents of the present invention may be formulated with conventional pharmaceutical or veterinary aids, for example, emulsifiers, fatty acid esters, gelling agents, stabilizers, antioxidants, osmolality adjusting agents, buffers, pH adjusting agents, etc., and may be in a form suitable for parenteral or enteral administration, for example injection or infusion or administration directly into a body cavity having an external escape duct, for example the gastrointestinal tract, the bladder or the uterus. The visualization materials of the present invention may be in any conventional pharmaceutical administration form, such as tablets, capsules, powders, solutions, suspensions, dispersions, syrups, suppositories, etc. However, solutions, suspensions and dispersions in physiologically acceptable carrier media, for example water for injections, that adhere to lumen walls, will generally be preferred.

For imaging of some portions of the body the most preferred mode for administering contrast agents is parenteral, e.g., intravenous administration. Parenterally administrable forms, e.g. intravenous solutions, should be sterile and free from physiologically unacceptable agents, and should have low osmolality to minimize irritation or other adverse effects upon administration, and thus the contrast medium should preferably be isotonic or slightly hypertonic. Suitable vehicles include aqueous vehicles customarily used for administering parenteral solutions, such as sodium chloride injection, Ringer's solution injection, dextrose injection, dextrose and sodium chloride injection, lactated Ringer's injection and other suitable solutions known in the art. The solutions may contain preservatives, antimicrobial agents, buffers and antioxidants conventionally used for parenteral solutions, excipients and other additives which are compatible with the chelates and which will not interfere with the manufacture, storage or use of products.

In one embodiment of the present invention, the contrast agent may conveniently comprise a gas-containing or gas-generating material, preferably in suspension in an aqueous carrier material and conjugated to one or more materials of i.e., an endothelin antagonist. The gas may take the form of microbubbles stabilized by a monolayer of a film-forming surfactant, or stabilized by a matrix material other than a surfactant. The materials may be, for example, coupled to such surfactant or matrix and may be bioactive or non-bioactive. There may be different targeting specificities and in one preferred embodiment are such as to interact with their receptors but not to fixedly bind the gas-containing vesicles.

Particularly useful contrast agents are compounds or materials that are visible under x-ray (i.e., heavy metals, iodine etc.). Regardless of the contrast agent employed, the visualization of body lumens (i.e., arteries, veins, biliary ducts, gastrointestinal tract, lyphatics, bronchi, etc.), by traditional procedures has a time limitation perhaps best exemplified by iodinated contrast agents visualized by x-ray radiation. The iodinated contrast is injected into the target lumen and the lumen contents (i.e., blood) are displaced for a brief period (i.e., seconds) of time. X-ray imaging is preformed rapidly to record the displacement process and to image the target lumen such that the presence or absence of disease processes can be diagnosed. The contrast agent is injected in bolus fashion and images must be obtained rapidly before the bolus has washed out. Repeated injections of contrast agent are common. Each repetition of the imaging procedure adds cost, increases the probability of a medical complication, and adds radiation exposure to both the patient and the provider. The present invention provides for the use of material that is imagable contrast material and has the functional capability of adhering to the lumen wall or surface for a period of time. The period of time should be of sufficient duration so as to provide visualization for the performance of invasive diagnostic procedures, therapeutic procedures or combined diagnostic and therapeutic procedures. The adherence time may vary. Adherence of contrast material may from about 1 or a few seconds to about 1 hour or more. Adherence time is preferably from about 10 seconds to about 5 minutes, and more preferably from about 15 seconds to about 2 minutes, with an even more preferable time of at least 30 seconds. Thus, the material of the invention functions in effect as a lumen paint providing extended time for visualization and thereafter being harmlessly removed from the body (i.e., biodegradation, excretion). The materials of the invention would adhere to the wall of the lumen so as to permit direct real-time visualization of the target lumen's tubular structure. Such real-time visualization is useful for both diagnostics and therapeutics, and is particularly well suited for invasive image guided procedures. For example, arteries and veins being treated with wires, balloons, catheters, stents and the like, would be visible under fluoroscopy while the procedure is performed. Success of such procedures requires careful placement and device manipulation within small areas. Such procedures, i.e., stent placement, would be greatly facilitated by the materials and methods of the present invention. Stents could be accurately placed since the lumen or vascular paint-like materials of the invention would allow real-time visualization of the diseased target area during stent placement and/or angiography.

The materials of the invention may be used for a variety of medical diagnostic procedures involving lumen visualization, including, but not limited to, the diagnosis of circulatory or digestive system conditions. For example, the present invention may conveniently be used to diagnose gastrointestinal (GI) bleeding. Gastrointestinal bleeding may involve the vomiting of blood (hematemesis), the passage of black tarry stool material (melena), or the passage of blood from the rectum (hematochezia) which suggests a lower GI source of bleeding. While anoscopy, flexible sigmoidoscopy, colonoscopy, nuclear medicine and other diagnostic procedures and tests known in the art are useful, the diagnosis of GI bleeding is often difficult. For example, if the bleeding rate is greater than about 0.5 mL/minute and conventional contrast materials are used, angiography may show extravasation of contrast medium.

The use of lumen-adhering contrast material would reduce radiation exposure to both the patient and operator during visualization procedures. Fluoroscopy generated images yield lower radiation doses to the patient and the operator than a digital angiographic acquisition. Iodine is a preferred contrast agent. Its efficacy is well known in the art. However, iodinated contrast agents are nephrotoxic. Large volumes of contrast agent are often needed during an inteventional procedure to confirm results of the procedure (i.e., to confirm stent or balloon position). The present invention would significantly lower the load of iodinated contrast to the kidneys. Accordingly, the present invention would allow for angiographic interventions to be performed safely on patients with abnormal renal function who would not otherwise be a candidate for this procedure.

Yet another benefit or advantage of the present invention is that the lumen paint-like contrast materials would predictably carry a decreased risk of allergic reaction to the contrast agent since very little of the contrast agent would come in contact with histiocytes in the lung vasculature. Also, the lumen paint-like contrast materials would reduce costs by decreasing the quantity of expensive contrast material needed to complete a procedure.

The lumen-adhering paint-like contrast materials of the invention may be an ionic or non-ionic contrast agent (i.e., an iodine-containing moiety) or may be a suitable contrast agent combined with a substance that binds to the walls of target lumens, preferably an endothelial binding substance. Contrast materials known in the art may conveniently be screened in vitro or in vivo for the functional capability of temporary adherence to lumen walls. For example, in an in vitro screen, artery sections may be obtained from animals, preferably small mammals, and more preferably mice or rates. Samples could be painted or otherwise applied to artery sections, flushed with saline or blood equivalent, and thereafter evaluated by visualization or chemical means for adherence characteristics (i.e., dwell time, etc.) of the sample to the artery wall. Samples may be in any convenient form (i.e., solid, liquid or gas, gel, or emulsion), may if necessary employ a pharmaceutically acceptable carrier, and should be physiologically acceptable. Contrast materials of the invention are preferably liquid or gel, and more preferably are aqueous solutions, mixtures or suspensions. Other bioassay screening systems known in the art may be employed. In vivo screening of materials with laboratory animal may be used. Samples of contrast materials are applied in vivo to target lumens of mammalian laboratory animals, preferably rats or mice, and visualized to identify contrast agents of the invention. Visualizing systems may be radiography systems, nuclear medicine systems, ultrasound systems, magnetic resonance systems or any combination of these systems.

In the case where a contrast material is combined with an endothelial binding substance, the endothelial binding substance(s) may be chemically bonded to the contrast moiety or otherwise chemically or physically adhered by any mechanism (i.e., chelated; encapsulated, etc.). The compositions of the invention are not limited by either the mechanism by which endothelial binding substance is bound or combined with a contrast molecule or moiety, or by the mechanism by which the lumen visualization materials of the invention adhere to lumen walls.

Endothelial binding by endothelial binding substance may be effected by any of the following substances or mechanisms, or any combinations thereof: ligand with affinity for PDGF receptors; integrin; phosphors tridentate; ionic or covalent bonding to the lumen wall (i.e., artery wall); van der Waal's forces; organic group with an affinity for endothelial cells; polymeric ligand; lipophilic ligand (i.e., PILH26-GL); ligand that binds to angiotensin receptor sites; ligand that enhances vessel wall permeability so contrast material seeps into lumen (i.e., artery) walls; ligand that is a junctional adhesion molecule (JAM); ligand that is magnetic so that contrast/ligand substance remains in vessel lumen by means of magnetic forces due to an external or internal magnetic field; ligand that is sticky by way of viscosity; ligand with an affinity to adhere to calcium ligand with an affinity to bind atheroma; ligand/contrast paint delivered through a standard angiographic catheter or via distal and/or proximal balloon occlusion or is applied to the vessel wall by a paint brush or the like attached to a wire, catheter or balloon; contrast material or molecule could also be a paramagnetic substance, visible by nuclear medicine imaging devices or ultrasound due to albumin or gas substrate; and ligand that could precipitate on contact with the lumen/vessel wall due to pH changes or due to molecular interactions between the paint molecule and endothelial cells.

The contrast materials providing continuous visualization of a body lumen are sometimes herein conveniently referred to as “vascular paint”. The vascular paint of the invention to be effective should adhere to the target vessel wall in sufficient amount so that the lumen surface may be visualized, and clearly visible under fluoroscopy as a preferred procedure. The contrast material or vascular paint should last at least one second, preferably 30 seconds or more, and should preferably biodegrade into harmless ingredients. Also, the vascular paint should not be nephrotoxic allergenic or stimulate atherogenesis.

Although the present invention describes in detail certain embodiments, it is understood that variations and modifications exist known to those skilled in the art that are within the invention. Accordingly, the present invention is intended to encompass all such alternatives, modifications and variations that are within the scope of the invention as set forth in the following claims.

What is claimed is:

1. A method for visualizing a body lumen comprising:
applying a contrast material to said body lumen, wherein said contrast material adheres completely or in part to the interior wall of said body lumen; and
visualizing said body lumen over a period time by a visualizing system.

2. The method of claim 1, wherein said method is continuous.

3. The method of claim 1, wherein said body lumen is selected from the group consisting of: the cardio-vascular system, the pulmonary system, the digestive system, the central nervous system, the reproductive system, and the excretory system.

4. The method of claim 1, wherein said body lumen is selected from the group of: arteries, veins, capillaries and lymphatic vessels.

5. The method of claim 1, wherein said contrast material is either an ionic or non-ionic contrast material.

6. The method of claim 5, wherein said contrast material comprises at least one heavy atom having an atomic weight of 30 or greater.

7. The method of claim 5, wherein said contrast material comprises at least one metal.

8. The method of claim 5, wherein said contrast material comprises iodine.

9. The method of claim 5, wherein said contrast material is selected from the group consisting of: metals, paramagnetic materials, high atomic number non-metal materials, radioisotopes, gases or gas precursors, chromatophores, fluorophores, electrical impedence materials, and any combinations thereof.

10. The method of claim 1, wherein said visualizing is over a period of time sufficient to permit the performance of image-guided invasive procedures selected from the group consisting of: diagnostic procedures, therapeutic procedures and any combinations thereof.

11. The method of claim 10, wherein said image-guided procedure is selected from the group consisting of: manipulation of wires, manipulation of balloons, manipulation of catheters, manipulation of stents, and diagnosis of gastrointestinal bleeding.

12. The method of claim 1, wherein said visualizing system is selected from the group consisting of: radiography systems, nuclear medicine systems, ultrasound systems, magnetic resonance systems and any combinations thereof.

13. The method of claim 1, wherein said contrast material further comprises an endothelial binding substance.

14. A contrast material for visualization of a body lumen wherein said contrast material exhibits the following characteristics:
(i) adheres to the interior wall of a target body lumen for a period of time sufficient to perform invasive image-guided procedures selected from the group consisting of: diagnostic procedures, therapeutic procedures and any combinations thereof, wherein said body lumen is selected from the group consisting of: the cardio-vascular system, the pulmonary system, the digestive system, the central nervous system, the reproductive system, and the excretory system;
(ii) provides visibility of said body lumen by a visualizing system sufficient to perform said invasive image-guided procedures, wherein said visualizing system is selected from the group consisting of: radiography systems, nuclear medicine systems, ultrasound systems, magnetic resonance systems and any combinations thereof; and
(iii) exits said body without causing either kidney toxicity, allergic reaction, or stimulation of atherogenesis.

15. The contrast material of claim 14, further comprising endothelial binding capability.

16. The contrast material of claim 14, wherein said contrast material is an ionic or non-ionic material.

17. The contrast material of claim 14, wherein said contrast material comprises at least one heavy atom having an atomic weight of 30 or greater.

18. The contrast material of claim 14, wherein said contrast material comprises iodine.

19. The contrast material of claim 14, wherein said contrast material comprises at least one metal.

20. The contrast material of claim 14, wherein said contrast material is selected from the group consisting of: metals, paramagnetic materials, high atomic number non-metal materials, radioisotopes, gases or gas precursors, chromatophores, fluorophores, electrical impedence materials, and any combinations thereof.

21. The contrast material of claim 13, wherein said contrast material is biodegradable.

22. A method for selectively binding contrast material to a lumen wall comprising:
administering an effective amount of a contrast material exhibits the following characteristics: (i) adheres to the interior wall of a target body lumen for a period of time sufficient to perform invasive image-guided procedures selected from the group consisting of: diagnostic procedures, therapeutic procedures and any combinations thereof, wherein said body lumen is selected from the group consisting of: the cardio-vascular system, the pulmonary system, the digestive system, the central nervous system, the reproductive system, and the excretory system; (ii) provides visibility of said body lumen by a visualizing system sufficient to perform said invasive image-guided procedures, wherein said visualizing system is selected from the group consisting of: radiography systems, nuclear medicine systems, ultrasound systems, magnetic resonance systems and any combinations thereof; and (iii) exits said body without causing either kidney toxicity, allergic reaction, or stimulation of atherogenesis; and
visualizing said lumen by a visualizing system.

23. The method of claim 22, wherein said period of time said contrast material adheres to said lumen wall is sufficient to permit the performance of image-guided invasive procedures selected from the group consisting of: diagnostic procedures, therapeutic procedures and any combinations thereof.

24. The method of claim 23, wherein said image-guided invasive procedure is selected from the group of: manipulation of wires, manipulation of balloons, manipulation of catheters, and manipulation of stents

25. The method of claim 22, wherein said visualizing system is selected from the group consisting of: radiography systems, nuclear medicine systems, ultrasound systems, magnetic resonance systems and any combinations thereof

26. The method of claim 22, wherein said contrast material is selected from the group consisting of: metals, paramagnetic materials, high atomic number non-metal materials, radioisotopes, gases or gas precursors, chromatophores, fluorophores, electrical impedence materials, and any combinations thereof.

27. The method of claim 22, wherein said contrast material comprises an endothelial binding substance.

28. The method of claim 22, wherein said contrast material comprises at least one metal.

29. The method of claim 22, wherein said contrast material comprises at least one heavy atom having an atomic weight of 30 or greater.

30. The method of claim 22, wherein said contrast material comprises iodine.
